(19) United States
(12) Patent Application Publication
(10) Pub. No.: US 2024/0161918 A1
(43) Pub. Date: May 16, 2024

(54) ASSET TRACKING SYSTEM WITH MEDICAL DEVICE INSPECTION SYSTEM

(71) Applicant: Clarus Medical, LLC, Minneapolis, MN (US)

(72) Inventors: Kristin Sundet PAVEK, Golden Valley, MN (US); Scott Allen SUNDET, Edina, MN (US); Andrew R. SUNDET, Excelsior, MN (US)

(73) Assignee: Clarus Medical, LLC, Minneapolis, MN (US)

(21) Appl. No.: 18/494,636

(22) Filed: Oct. 25, 2023

Related U.S. Application Data
(60) Provisional application No. 63/380,767, filed on Oct. 25, 2022.

(51) Int. Cl. G16H 40/40

(52) U.S. Cl. CPC G16H 40/40

(57) ABSTRACT

An asset tracking system includes a medical device inspection system. In some embodiments the asset tracking system includes asset tracking software and a medical device inspection system. The medical device inspection system supplements the asset tracking software to provide additional functionality for inspection of a medical device using a medical device inspection system.

### CROSS-REFERENCE TO RELATED APPLICATION(S)

This application claims the benefit of U.S. Provisional Patent Application No. 63/380,767, filed on Oct. 25, 2022, entitled ASSET TRACKING SYSTEM WITH MEDICAL DEVICE INSPECTION MODULE, the disclosure of which is hereby incorporated by reference in its entirety. To the extent appropriate, a claim of priority is made to the above-listed application.

### BACKGROUND

Healthcare providers utilize many different tools, instruments, and supplies. Asset tracking systems are available that allow healthcare providers to track such assets. Tracking of assets is more than just inventory and location tracking. It also involves managing and tracking the status of assets as they move through various workflows in order to ensure that the assets will be available and ready when needed.

### SUMMARY

In general terms, this disclosure is directed to an asset tracking system. In some embodiments, and by non-limiting example, the asset tracking system includes a medical device inspection system.

One aspect is an asset tracking system comprising: at least one processing device; and at least one memory device storing asset tracking software and a medical device inspection system, wherein the medical device inspection system, when executed by the processing device, supplements the asset tracking software to provide additional functionality for inspection of a medical device using a medical device inspection system.

Another aspect is an asset tracking system comprising: at least one processing device; and at least one memory device, the memory device storing data instructions that, when executed by the processing device, cause the asset tracking system to: identify a medical device; retrieve a workflow for the medical device; and track progress of the medical device through the workflow, wherein the workflow comprises at least one inspection step involving inspection of the medical device using an inspection scope.

A further aspect is a computer readable storage device storing data instructions for a medical device inspection system, which when the data instructions are executed by a processing device, cause the computing device to: retrieve at least one reference image associated with the medical device; and generate a user interface including the reference image and an inspection image from the medical device inspection scope within an asset tracking software application.

Yet another aspect is a method of operating an asset tracking system, as disclosed herein.

### BRIEF DESCRIPTION OF THE DRAWINGS

FIG. 1 is a schematic block diagram illustrating an example asset management system according to the present disclosure.

FIG. 2 is a schematic block diagram illustrating an example asset tracking system of the asset management system shown in FIG. 1.

FIG. 3 is a schematic block diagram illustrating an example asset tracking software with an example medical device inspection system.

FIG. 4 is a flow chart illustrating an example method of tracking assets using a medical device inspection system.

FIG. 5 illustrates an example medical device selection interface of the asset tracking software.

FIG. 6 illustrates an example workflow user interface presented during the example workflow shown in FIG. 3, associated with a medical device.

FIG. 7 illustrates an example user interface of a medical device inspection system.

FIG. 8 illustrates an example computing device.

FIG. 9 is a schematic block diagram illustrating an example medical device inspection system, including an example medical device inspection station.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

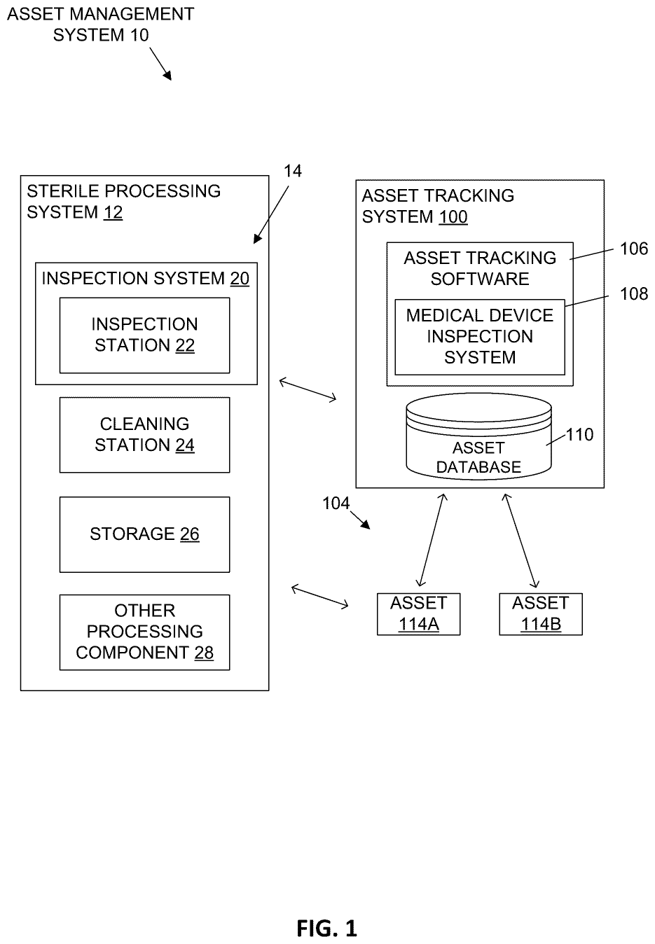

FIG. 1 is a schematic block diagram illustrating an example asset management system 10. In this example, the asset management system 10 includes a sterile processing system 12 and an asset tracking system 100. A plurality of assets 114 (including assets 114A and 114B) are shown. The example sterile processing system 12 includes one or more components 14, such as an inspection system 20 (including an inspection station 22), a cleaning station 24, a storage 26, and one or more other processing components 28. The example asset tracking system 100 includes an asset tracking software 106, a medical device inspection system 108, and an asset database 110.

The example asset management system 10 operates to manage a plurality of assets 114. For example, the asset management system 10 can operate to manage assets for a healthcare facility or group of healthcare facilities. Examples of assets 114 are medical devices. Management of assets can involve a variety of different operations, including, for example, tracking locations of assets, cleaning assets, managing statuses of assets, and the like.

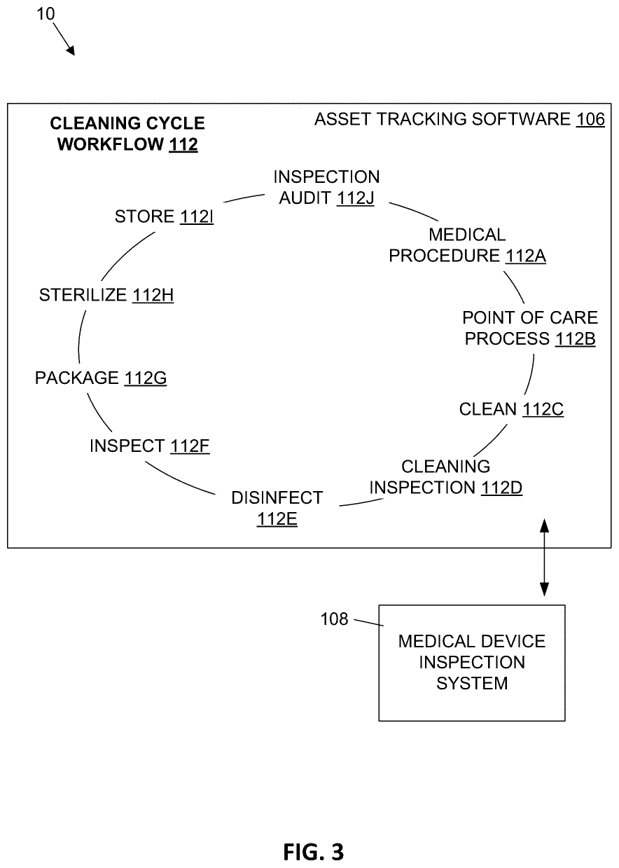

In some embodiments the management system 10 operates to manage the assets through a workflow. The workflow can be defined by the healthcare facility, a healthcare provider, a healthcare network, by an asset manufacturer, by the asset tracking system 100, or the like, and such workflows can be changed or updated periodically. The workflows can include a series of processes that the asset 114 will go through during its working lifecycle within a facility. For example, an asset 114 may need to be inspected by an inspection system 20, cleaned at a cleaning station 24, stored in the storage 26, or otherwise processed by one or more other processing components 28. Further, in many embodiments the asset 114 may be used for its intended purpose at a point of use (such as a surgical instrument being used for a surgical procedure, or a medical device being used for a medical procedure). Other processes can also be used in various workflows, such as service and/or repair processes, and a wide variety of other possible processes. An example workflow 112 is illustrated in FIG. 3.

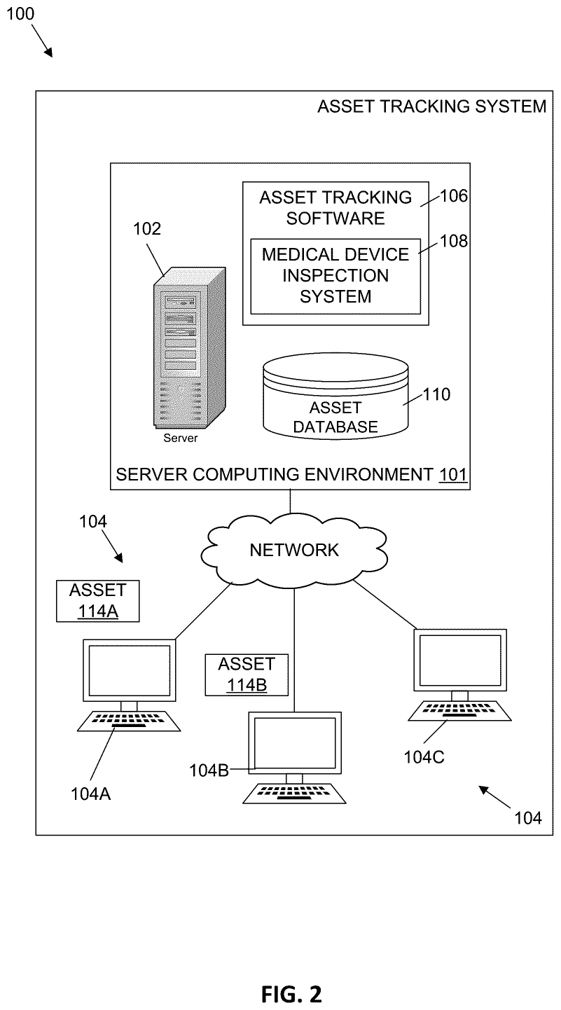

An example of the asset tracking system 100 will now be discussed in further detail with reference to FIG. 2.

FIG. 2 is a schematic block diagram illustrating an example asset tracking system 100. In this example, the asset tracking system 100 includes a server computing environment 101 and a plurality of client computing devices 104 (including 104A, 104B, and 104C). The server computing environment 101 includes a server computing device 102 with asset tracking software 106. The asset tracking software 106 includes a medical device inspection system 108. The server computing environment 101 also includes an asset database 110. Several examples of assets (assets A and B) are also shown.

The asset tracking system 100 operates to track the assets 114 (including in this example, asset 114A and asset 114B). The asset tracking system 100 may track the assets 114 within a healthcare system. A healthcare system can include one or more healthcare providers. In addition to tracking inventory and asset locations, the asset tracking system can also track workflows associated with certain assets 114. For example, some medical devices are reusable. But, often such medical devices must undergo certain workflows, including various processing steps, before they can be reused. Examples of such processing steps include cleaning and sterilization. The asset tracking system 100 stores information on such workflows, and tracks the assets 114 as they progress through the process.

Healthcare provider personnel interact with the asset tracking system through client computing devices 104. For example, an application or web interface is provided on the client device, which retrieves information from the server computing device 102. The personnel provide information and updates to the asset tracking system on the status of the assets 114.

The asset tracking software 106 and medical device inspection system 108 are illustrated and described in further detail with reference to the following figures. Although they are shown in FIG. 2 as residing in the server computing environment 101 and the server computing device 102, either one of, or both of the asset tracking software 106 and the medical device inspection system 108 can alternatively reside on computing devices 104 operated by the healthcare provider personnel. Or, in another possible embodiment, aspects of the asset tracking software 106 and medical device inspection system 108 may reside on both the server 102 and the client computing devices 104. Similarly, the asset database 110 may reside on server 102, another cloud storage device, any one of the computing devices 104, or combinations thereof.

The example shown in FIG. 2 depicts the medical device inspection system 108 as being a component of the asset tracking software 106. In some embodiments the medical device inspection system 108 is a plugin to the asset tracking software 106. Other variations are also possible. For example, in some embodiments the medical device inspection system 108 and the asset tracking software 106 are separate from one another. In some embodiments the medical device inspection system 108 and the asset tracking software 106 can communicate with one another, such as through the server 102, or through data communication across a network, such as a local area network or the Internet. In a more specific example, one or more application programming interfaces (APIs) can be used on one or both of the medical device inspection system 108 and the asset tracking software 106 for data communication therebetween or with other computing devices.

Another embodiment includes a medical device inspection software, wherein the medical device inspection software includes the medical device inspection system 108 and the asset tracking software 106. In other words, the medical device inspection software can provide both functionalities.

FIG. 3 is a schematic block diagram illustrating an example workflow 112 associated with a medical device. In this example, the workflow 112 is managed by the asset tracking system 100, including the asset tracking software 106 using the medical device inspection system 108.

The asset tracking software 106 operates to identify and track certain workflows 112 associated with assets 114 (FIG. 1). An example of an asset 114 is a medical device. Different workflows are appropriate for different assets 114. Therefore, in some embodiments the asset tracking software 106 can store and manage the official workflows of a healthcare provider that define what steps the medical devices should go through. In some embodiments, the operations of the asset tracking software 106 are enhanced by the medical device inspection system 108, which provides extended functionality to the asset tracking software 106 relating to medical device inspection operations. In some embodiments the medical device inspection system 108 is or includes an interior inspection system, operable to inspect interiors of medical devices.

As discussed herein, although the medical device inspection system 108 is shown in FIG. 3 as being separate from the asset tracking software 106, in other configurations the medical device inspection system 108 can be part of the asset tracking software 106, such as by having the operations of the medical device inspection system 108 built into the asset tracking software 106. In yet another possible embodiment, the workflow 112 can be managed directly by the medical device inspection system 108, such that the operations of the asset tracking software 106 are built into the medical device inspection system 108.

An example workflow 112 is illustrated in FIG. 3 for a particular asset 114, where the asset 114 is a medical device. As discussed, a wide variety of possible workflows 112 can be defined for different assets 114. This example workflow 112 is a cleaning cycle workflow for an example medical device.

The example workflow 112 includes a plurality of workflow stages 112A-112J. The example workflow 112 begins with the medical device being used during a medical procedure 112A, also sometimes referred to as the “point of use” or “point of care”. An example of a medical procedure is a surgical procedure, though many medical devices can be used for many other purposes. Use of a medical device in a medical procedure, such as surgery, exposes the medical device to contaminants. Therefore, a cleaning cycle workflow 112 is defined for the medical device in the asset tracking software 106, in order to clean the medical device before it is used with in another medical procedure with another patient. The medical procedure 112A involves using the medical device for its intended purpose.

After the medical procedure 112A, the workflow 112 includes a point of care processing stage 112B. In this example, the workflow 112 defines some initial processing steps that should take place while the medical device is still at the point of care. An example is wiping off the medical device and placing it into some temporary packaging for transportation to the next workflow stage.

The next workflow stage defined by the workflow 112 is the cleaning stage 112C. In some embodiments the cleaning stage 112C is performed by a cleaning station 24, shown in FIG. 1. The cleaning stage 112C involves processing the medical device through a thorough cleaning process. The cleaning stage 112C can include manual, mechanical, and/or automated cleaning processes, which often involve the use of water and detergents. In some embodiments the cleaning stage 112C involves certain machinery or appliances, such as ultrasonic cleaners, washer-disinfectors, cart washers, endoscopic reprocessors, or other cleaning machines.

The workflow 112 then proceeds to a cleaning inspection stage 112D. During the cleaning inspection stage 112D, the operations of the cleaning stage 112C are checked to confirm that they were successful and thorough. In some embodiments the medical device inspection system 108 is used during this stage, which may also involve the use of the inspection system 20.

In some embodiments the exterior of the medical device is inspected. This can be performed manually by an operator, such as by visual inspection, with results being entered into the asset tracking system 100. Exterior inspection can also be performed using the inspection system 20, such as using a camera. For example, an exterior image of the medical device can be captured by the camera and then processed by the medical device inspection system 108 to check for possible abnormalities. Some embodiments utilize an exterior inspection device for inspecting the exterior of the medical device. An example of the exterior inspection device is described in applicant's co-pending application titled Medical Device Inspection System With External Inspection Device, filed on Oct. 25, 2023, U.S. application Ser. No. 18/494,573, which claims priority to U.S. Application No. 63/380,766, filed on Oct. 25, 2022, titled Medical Device Inspection System With External Inspection Device, the disclosures of which are hereby incorporated by reference in their entireties.

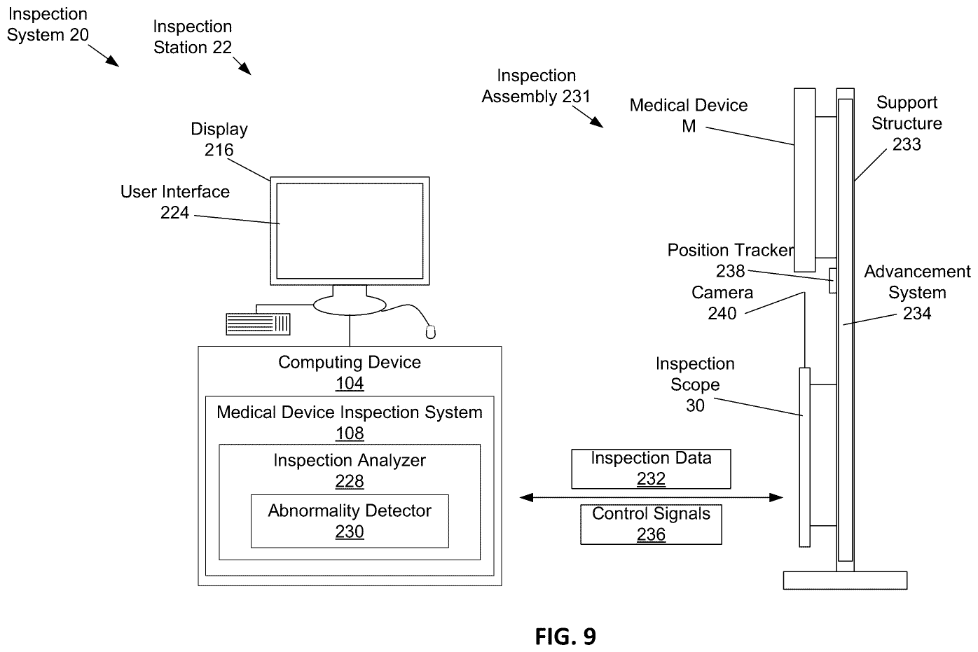

In some embodiments the cleaning inspection stage 112D involves inspecting the interior of the medical device. For this, an interior inspection device is used, such as the example inspection scope 30 shown in FIG. 9. Interior images of the medical device are captured by the inspection scope 30 and then processed by the medical device inspection system 108 to check for possible abnormalities.

If determined in the cleaning inspection stage 112D that the medical device is not adequately cleaned, the workflow 112 can define that the medical device is then returned to the cleaning stage 112C for further cleaning.

Once the medical device has passed the cleaning inspection stage 112D, the workflow 112 then advances the medical device to the disinfecting stage 112E, which is designed to reduce or eliminate pathogenic microorganisms from the medical device. In some embodiments the disinfecting stage 112E is performed by a cleaning station 24, shown in FIG. 1. In some examples the disinfecting stage 112E includes manual or mechanical disinfection. Manual disinfection typically involves wiping or soaking the medical device in liquid chemicals such as disinfectant solutions. Mechanical disinfection includes the use of machines to disinfect with water, detergent, and heat. In some workflows the cleaning and disinfecting stages are combined into a single stage. The disinfecting stage 112E can also involve the application of ultraviolet (UV-C) light or hydrogen peroxide vapor. In some embodiments the UV-C light is applied using an interior inspection device, such as a borescope, configured to emit UV-C light. In some embodiments the medical device inspection system 108 is used during the application of UV-C light using the interior inspection device.

The workflow 112 then proceeds to another inspecting stage 112F. During the inspecting stage 112F, the operations of the cleaning stage 112C and disinfecting stage 112E are checked to confirm that they were successful and thorough, and may also check that no residual moisture or chemicals remain. Any other abnormalities can also be identified during the inspection stage 112F. In some embodiments the medical device inspection system 108 is used during this stage, which may also involve the use of the inspection system 20. In some embodiments the inspection stage 112F is the same or similar to that of the cleaning inspection stage 112D, described above.

If determined in the inspection stage 112F that the medical device is not adequately cleaned or disinfected, the workflow 112 can define that the medical device is then returned to stages 112C and/or 112E for further processing. If any other abnormalities are identified, then appropriate remedial action may be defined by the workflow 112.

Once the inspection confirms that the medical device is properly cleaned and disinfected and ready for the next medical procedure 112A, the workflow 112 advances the medical device to the packaging stage 112G. During the packaging stage 112G, the medical device is enclosed in specialized packaging materials, such as sterile wraps, pouches, or containers. The packaging acts as a barrier to protect the medical device from contamination, so that it remains sterile until the next medical procedure 112A.

Next, the workflow 112 advances to the sterilization stage 112H. During sterilizing, the medical device undergoes further processing in an effort to destroy any remaining microbial life, including bacterial spores. The sterilization stage can be performed by a variety of machines or devices, depending on the particular medical device and the particular workflow 112. Example sterilization processes can include the use of steam autoclaves, ethylene oxide (EtO) sterilizers, hydrogen peroxide gas plasma sterilizers, ozone sterilizers, dry heat sterilizers, and the like.

Following the sterilization stage 112H, the medical device is then placed into storage 112I. Cleanliness and sterility are maintained by the packaging and storage. Even when in storage, the asset tracking software 106 can track the status and location of the medical device to ensure that it is ready and available for another future medical procedure 112A.

In the illustrated workflow 112, an additional inspection audit stage 112I is performed. In this example, the inspection audit stage 112I is an additional quality control measure designed to ensure the medical device remains clean, sterile, and ready for the next medical procedure 112A. The inspection audit stage 112I can include, for example, an integrity check of the packaging (i.e., for tears, punctures, dampness, etc.), checking of sterility indicators, checking of storage conditions, checking expiration dates, and the like.

In some embodiments, the workflow 112 involves the use of a medical device inspection system 108. The medical device inspection system 108 is utilized during one or more medical device inspection stages of the workflow 112. The inspection process can be performed using a medical device inspection system 20 to inspect the medical device. For example, the inspection process can check the medical device for abnormalities. Abnormalities can include, for example, debris, damage (e.g., gouges, kinks, cracks), discoloration, moisture (e.g., water droplets), contaminants (e.g., biofilms or biological material), and the like. If any abnormalities are identified, then further inspection or processing of the medical device may be warranted. Additional processing can include, for example, repeating the cleaning stage 112C or disinfecting stage 112E, obtaining a replacement part, or sending the device out for service. Many other additional stages are possible in other workflows 112.

An example of a medical device inspection system is a medical device inspection scope 30 (FIG. 9). An example of an inspection scope is a borescope, such as a fiber scope. The inspection scope typically includes a light source and a camera. An example of the inspection scope has a long narrow fiber that can be inserted and removed through the center of the medical device, and operates to capture images of the interior of the medical device. Examples of inspection scopes are disclosed in various patent applications by Clarus Medical, LLC including US 2019/0224357, filed on Jan. 22, 2019; US 2019/0282327, filed on Feb. 19, 2019; US 2022/0080469, filed on Sep. 10, 2021; and US 2022/0240767, filed on Feb. 3, 2022, the disclosures of which are hereby incorporated by reference in their entireties.

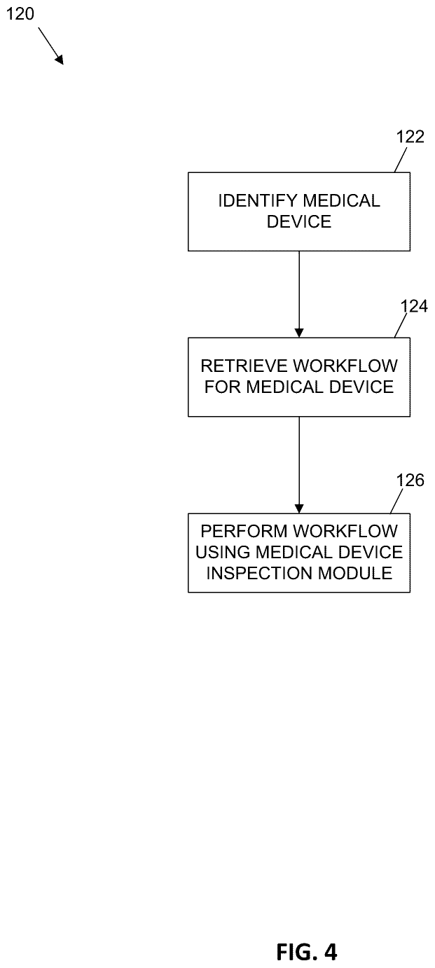

FIG. 4 is a flow chart illustrating an example method 120 of tracking assets using a medical device inspection system 108. In this example, the method 120 includes operations 122, 124, and 126.

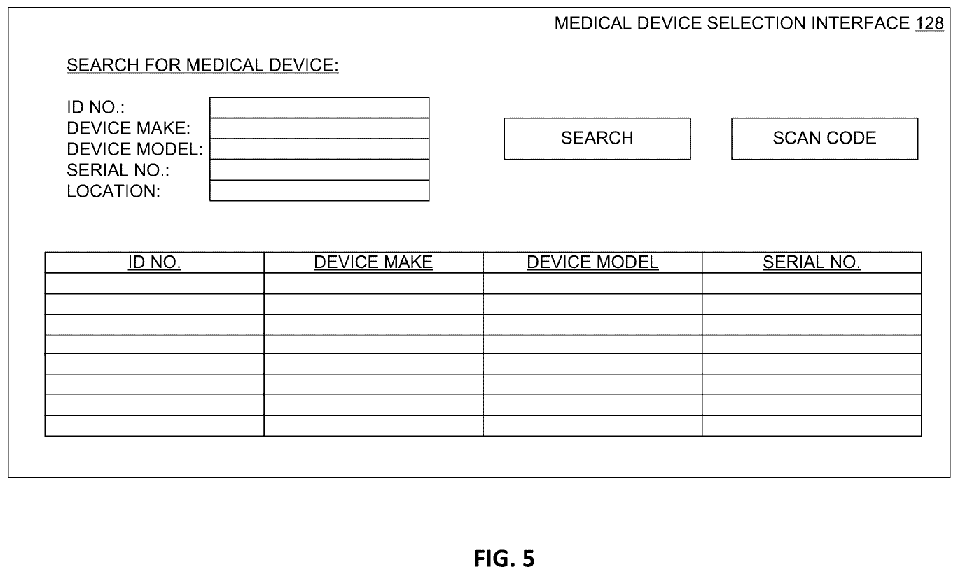

The operation 122 is performed to identify the medical device. An example is illustrated and described in further detail with reference to FIG. 5.

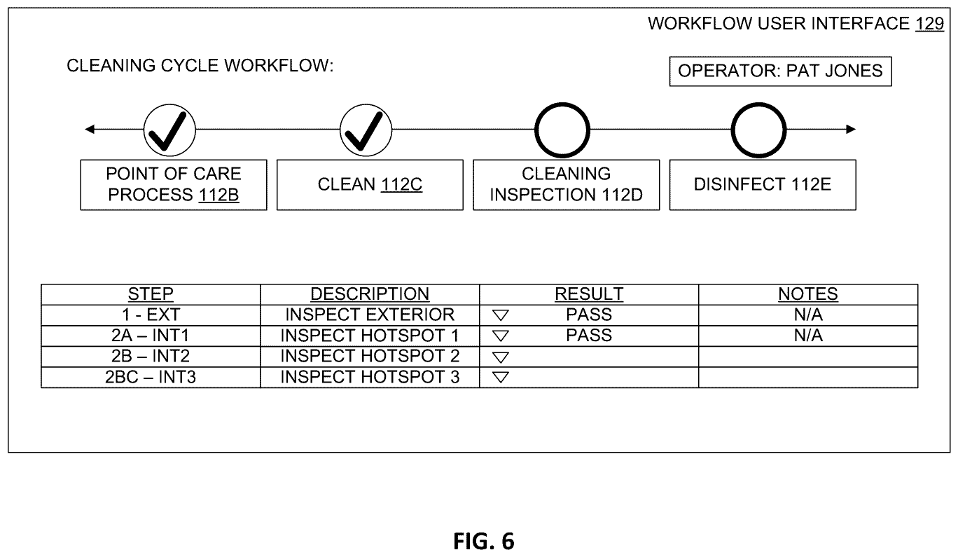

The operation 124 is then performed to retrieve a workflow for the identified medical device. An example workflow is illustrated and described in further detail with reference to FIG. 6. One example of a workflow is Instructions For Use (IFU). In the United States, the FDA requires all medical devices to have instructions for use. Such instructions for use often define workflows such as cleaning processes to be used for the particular medical device.

The operation 126 is then performed to perform the workflow. In some embodiments the performance of the workflow involves using the medical device inspection system 108. An example of a workflow is illustrated and described in further detail herein with reference to FIG. 3.

FIG. 5 illustrates an example medical device selection interface 128 of the asset tracking software 106. The medical device selection interface is used by an operator to identify the medical device records within the asset tracking software 106. For example, when an operator wishes to perform a stage of a workflow, the operator can use the medical device selection interface to pull up the records for the particular medical device in the asset tracking system, for review or updating.

The identification can be performed by entering in medical device information, scanning the medical device information, or by looking up the medical device using one or more search or lookup processes. As one example, the operator can enter a manufacturer and model number, and/or a serial number or other medical device identification number. As another example, the operator can scan the medical device with a barcode reader, and RFID reader, or a digital camera (e.g., to read a QR code or text, or to perform object recognition). In another example, the operator can perform a search of the asset database to find the relevant medical device. In another example, the operator can search for medical devices associated with a particular case number.

In this example, once a search has been conducted or a code scanned, the resulting list of medical devices is displayed. The operator can then scroll through the list and select the medical device from the list.

The asset tracking software 106 can maintain a wide variety of data regarding medical devices. Some example fields of data that can be stored by the asset tracking system include one or more of (but is not limited to), device make, device model, serial number, time/date/technician performing each step, status, percent confidence in analysis, location of device, procedure device used on, surgeon using the device, patient medical device was used on, date of last repair, vendor of device, cleaning completed, inspection completed, sterilization completed, time/date of sterilization, temperature/metadata on previous steps, make/model/type of brush (or other cleaning instrument) used when cleaning, soap/chemical used, concentration of soap/chemical used, metadata for steam sterilizing settings, dosage of UV-C applied during inspection or cleaning, images captured at each of the hotspots, reviewed by staff, and speed/length of time/quality of inspection. In some embodiments the medical device selection interface can allow for searching of, display of, and identification of medical devices using any one or more of these example fields, or a variety of other data fields.

FIG. 6 illustrates an example workflow user interface 129 presented during an example workflow 112 (shown in FIG. 3) associated with a medical device. In some embodiments, the workflow user interface 129 is generated by the asset tracking system 100, such as by the asset tracking software 106 or by the medical device inspection system 108.

After the medical device has been identified in operation 122, such as using the example user interface 128 shown in FIG. 5, the workflow 112 associated with the medical device can be retrieved. In one example, the workflow 112 is retrieved from the asset database 110, shown in FIG. 2. Each asset (e.g., medical device) is associated with one or more workflows. In some embodiments different workflows can be available for different statuses of the medical device. For example, after use, the medical device may need to go through the cleaning cycle workflow 112.

The example user interface 129 includes a workflow preview region that displays certain stages of the workflow 112 for the selected medical device. In this example, the workflow preview region displays stages 112B-112E of the workflow 112. The workflow preview region provides a brief description of the stage, as well as a status. In this example, each stage is associated with a graphical icon showing whether or not that stage has been completed. In this example, stages 112B (point of care processing) and 112C (cleaning) have been completed, and the medical device is currently undergoing the cleaning inspection stage 112D.

At each stage of the workflow 112, certain actions are performed and certain data is collected. The workflow shown is just illustrative, and many other workflows are possible.

The workflow 112 can be displayed by the asset tracking software 106 or medical device inspection system 108, so that the personnel can see what steps need to be taken, and the current status of the medical device in the workflow. Further, the asset tracking software 106 or medical device inspection system 108 can also provide detailed instructions for each stage (such as represented by the list of steps, and description of each step), and collect data for each stage such as test results, and notes. Additional data can be collected and stored in other embodiments, including inspection data and images captured during inspection.

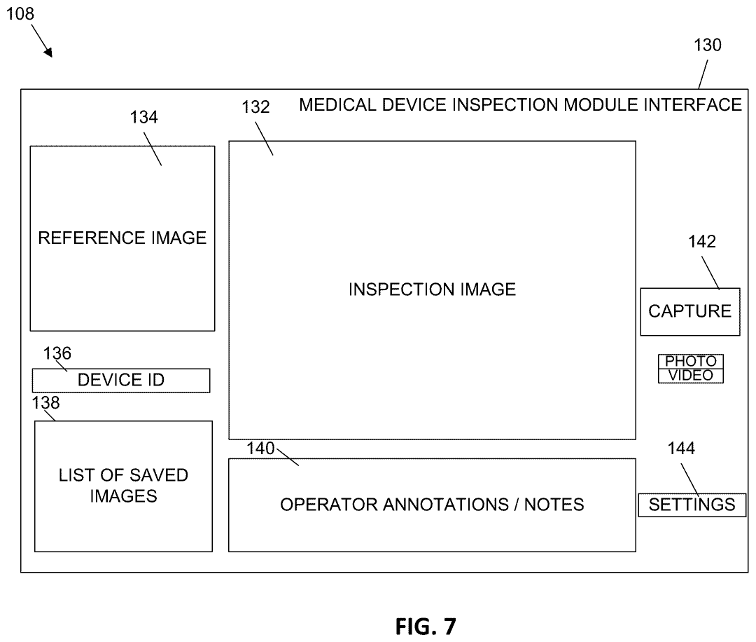

FIG. 7 illustrates an example user interface 130 of the medical device inspection system 108. In this example, the user interface includes an inspection image display 132, a reference image display 134, a device ID 136, a list 138 of saved images, operator annotations or notes 140, a capture button 142, and a settings button 144. This display is merely for illustrative purposes, and other user interfaces can have more or fewer components than shown here.

In some embodiments the interface 130 is the user interface that an operator can interact with while using a medical device inspection system, such as medical device inspection scope. In some embodiments the medical device inspection system 108 provides the interface 130 that includes imagery and data from the medical device inspection system.

For example, the inspection image display 132 shows the most recent image received from the medical device inspection system, while inspecting the medical device. The image may be a still image or may be a frame from a video feed.

In some embodiments the interface 130 also presents to the operator one or more reference images. The reference images can be retrieved from the asset database for the particular medical device. The reference images can show, for example, what the original medical device looks like when clean and fully functional (absent any abnormalities). In this way the operator can compare the inspection image 132 with the reference image to check for any abnormalities or other differences between the inspection image 132 and the reference image 134. In another possible example, the reference images can show examples of abnormalities, so that the operator can be on the lookout for such features. In another possible example, the reference images can include historical imagery from the same medical device that is currently being processed. This can be useful to compare the inspection image 132 with the previous set of images that were taken to see whether anything has changed. Similarly, imagery over a period of time can be viewed, such as to see the progression of abnormalities, such as wear, damage, buildup of films or contaminants, rusting components, and the like.

The interface 130 can display information about the medical device currently being processed (such as the device identifier 136), and/or about the medical device inspection system currently being used.

The interface 130 can include a list 138 of images that have already been saved during the current inspection process. The saved images can be reviewed by the operator if desired.

The interface 130 can also be configured to receive operator annotations or notes. The operator can provide input identifying any status changes in the medical device, any noted abnormalities, the completion of a workflow processing step, or make any general notes or observations.

A capture button 142 is provided for the operator to select when an image should be captured and saved. For example, if an abnormality is detected in the inspection image 132, the capture button is selected to save that image. The capture button can alternatively be used to capture and save a video recording. In some embodiments the operator can toggle between image or video capturing modes.

In some embodiments the user can adjust one or more settings via the settings button 144.

In some embodiments the medical device inspection system 108 provides detailed step-by-step instructions to the operator that guide the operator through the completion of the workflow step. The instructions may also be shown in the interface 130.

In some embodiments, the asset database can store a list of landmarks for the medical device, such as a list of hotspots. The list of hotspots identifies particular parts of the medical device where abnormalities are most likely to be found. A hotspot might be a component of the medical device that tends to wear out and may need to be replaced. A hotspot might also be a location at which contaminants are likely to accumulate. A hotspot might also be a location at which damage is more likely to occur, such as a point along a flexible member where kinking or cracking is more likely. The list of hotspots can be provided as a helpful guide to the operator, or can be presented as a mandatory checklist of regions that must be carefully evaluated by the operator. In such a case, the interface 130 can guide the operator through the evaluation of each hot spot, and function to record data about the status of each (either automatically or based on user input or a combination of both).

In some embodiments the interface displays other data, such as a position of the medical device inspection scope within the medical device. The position information can be helpful for the operator to locate the hotspots, and also to help the operator document the location of possible abnormalities. Position information can also be stored along with the saved images. Similarly, time can be displayed and stored. Data relating to the images (including abnormalities, operator annotations, device ID, position information, time information, etc.) can be stored in a variety of ways including, as data in the asset database, as metadata in the image (or video) file, as part of the file name, or in any other way that the data can be associated with the images. Such information may also be stored in the asset database records. For example, as data associated with the completion of certain workflow steps, or to document and validate that such steps were completed.

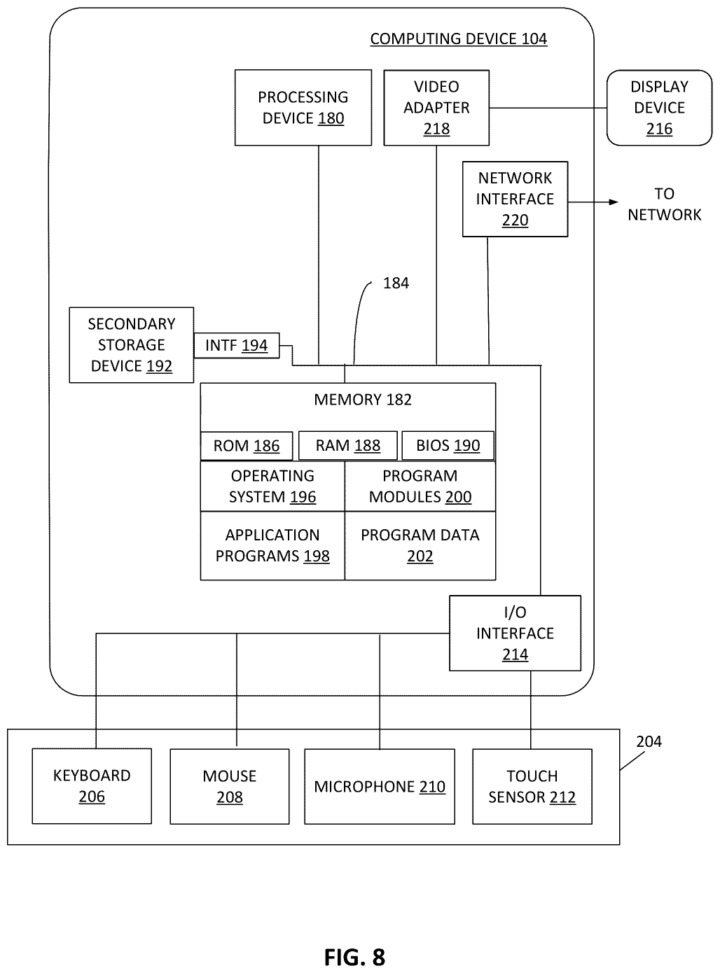

FIG. 8 illustrates an exemplary architecture of a computing device that can be used to implement aspects of the present disclosure, including the server computing device 102 or any of the plurality of client computing devices 104 (104A, 104B, or 104C) disclosed herein. The computing device illustrated in FIG. 8 can be used to execute the operating system, application programs, and software modules (including the software engines) described herein. By way of example, the computing device will be described below as the computing device 104. To avoid undue repetition, this description of the computing device will not be separately repeated herein for each of the other computing devices, including server computing device 102, but such devices can also be configured as illustrated and described with reference to FIG. 8.

The computing device 111 includes, in some embodiments, at least one processing device 180, such as a central processing unit (CPU). A variety of processing devices are available from a variety of manufacturers, for example, Intel or Advanced Micro Devices. In this example, the computing device 111 also includes a system memory 182, and a system bus 184 that couples various system components including the system memory 182 to the processing device 180. The system bus 184 is one of any number of types of bus structures including a memory bus, or memory controller; a peripheral bus; and a local bus using any of a variety of bus architectures.

Examples of computing devices suitable for the computing device 111 include a server computer, a desktop computer, a laptop computer, a tablet computer, a mobile computing device (such as a smart phone, an iPod® or iPad® mobile digital device, or other mobile devices), or other devices configured to process digital instructions.

The system memory 182 includes read only memory 186 and random-access memory 188. A basic input/output system 190 containing the basic routines that act to transfer information within computing device 111, such as during start up, is typically stored in the read only memory 186.

The computing device 111 also includes a secondary storage device 192 in some embodiments, such as a hard disk drive, for storing digital data. The secondary storage device 192 is connected to the system bus 184 by a secondary storage interface 194. The secondary storage devices 192 and their associated computer readable media provide nonvolatile storage of computer readable instructions (including application programs and program modules), data structures, and other data for the computing device 111.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 192 or memory 182, including an operating system 196, one or more application programs 198, other program modules 200 (such as the software engines described herein), and program data 202. The computing device 111 can utilize any suitable operating system, such as Microsoft Windows™, Google Chrome™, Apple OS, and any other operating system suitable for a computing device.

In some embodiments, a user provides inputs to the computing device 111 through one or more input devices 204. Examples of input devices 204 include a keyboard 206, mouse 208, microphone 210, and touch sensor 212 (such as a touchpad or touch sensitive display). Other embodiments include other input devices 204. The input devices are often connected to the processing device 180 through an input/output interface 214 that is coupled to the system bus 184. These input devices 204 can be connected by any number of input/output interfaces, such as a parallel port, serial port, game port, or a universal serial bus. Wireless communication between input devices and the interface 214 is possible as well, and includes infrared, BLUETOOTH® wireless technology, 802.11a/b/g/n, cellular, or other radio frequency communication systems in some possible embodiments.

In this example embodiment, a display device 216, such as a monitor, liquid crystal display device, projector, or touch sensitive display device, is also connected to the system bus 184 via an interface, such as a video adapter 218. In addition to the display device 216, the computing device 111 can include various other peripheral devices (not shown), such as speakers or a printer.

When used in a local area networking environment or a wide area networking environment (such as the Internet), the computing device 111 is typically connected to the network through a network interface 220, such as an Ethernet interface. Other possible embodiments use other communication devices. For example, some embodiments of the computing device 111 include a modem for communicating across the network.

The computing device 111 typically includes at least some form of computer readable media. Computer readable media includes any available media that can be accessed by the computing device 111. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 111. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 8 is also an example of programmable electronics, which may include one or more such computing devices, and when multiple computing devices are included, such computing devices can be coupled together with a suitable data communication network so as to collectively perform the various functions, methods, or operations disclosed herein.

FIG. 9 is a schematic block diagram illustrating an example medical device inspection system, including an example medical device inspection station 22. The example inspection system 20 includes a computing device 104 and an inspection assembly 231. The example computing device 104 includes a medical device inspection system 108. In this example, the medical device inspection system 108 includes an inspection analyzer 228 with an abnormality detector 230. The example computing device 104 also includes a display device 216, which displays a user interface 224. The example inspection assembly 231 includes a support structure 233, an advancement system 234, a position tracker 238, and an inspection scope 30 including a camera 240. The inspection assembly 231 is shown supporting a medical device M thereon. Inspection data 232 is generated by the inspection system and can be communicated between the inspection assembly 231 and the computing device 104.

In some embodiments, the inspection station 22 is used as part of the method 120 illustrated and described in reference to FIG. 4. Examples of the user interfaces 224 are illustrated and described in reference to FIGS. 5-7. In some embodiments, the computing device 104 includes some or all of the components illustrated and described in reference to FIG. 8. In some embodiments, the computing device 104 communicates with other computing devices via a network, for example, the computing device 102 of the server computing environment 101 illustrated and described in reference to FIG. 2.

This example illustrates the medical device inspection system 108, inspection analyzer 228, and abnormality detector 230 residing on computing device 104. However, as shown in FIG. 2, the medical device inspection system 108 can also reside on a server computing environment (or other computing devices) in other embodiments. Further, in some embodiments multiple computing devices (i.e., 104 and 102) can cooperate to collectively perform the functions of the medical device inspection system 108. It has also previously been explained that the medical device inspection system 108 (which in FIG. 9 is shown to include the inspection analyzer 228 and/or abnormality detector 230) can be part of the asset tracking system 100 and software 106, and in another embodiment the asset tracking system 100 and software 106 can be integrated into the medical device inspection system 108.

In some embodiments the inspection assembly 231 includes a support structure 233, for supporting the inspection scope 30 and the medical device M. The support structure 233 can take a variety of possible forms, and typically includes at least a frame or other housing that supports and optionally guides movement of various components of the inspection station 22 with respect to one another. In some embodiments the support structure 233 is a vertical support structure that can support one or more of, or portions of, the medical device M or the inspection scope 30 in a vertical orientation. An advantage of the vertical support structure configuration is that it can reduce table or floor space, for example. In other embodiments the support structure 233 includes a horizontal support structure to support in a horizontal orientation.

In some embodiments, the inspection scope 30 includes a camera 240 for visually inspecting the medical device M. In some embodiments the inspection scope 30 transfers the inspection data 232 to the computing device 104.

As discussed herein, in some embodiments the inspection assembly 231 further includes an exterior inspection device, which may be supported by the support structure 233 and movable relative to the medical device by the advancement system 234.

The advancement system 234 is configured to move the inspection scope 30 relative to the medical device M. In some embodiments, the advancement system 234 is motorized to move the inspection scope 30 or medical device M.

As discussed above, in some embodiments, the advancement system 234 is configured to operate automatically. For example, the advancement system 234 may include a robotic arm or auto feed device which advances the inspection scope 30 through the medical device M. Other motorized, mechanical, manual methods can be used in different embodiments and are disclosed herein. In some embodiments, the advancement system 234 the inspection scope 30 captures inspection data which is processed by a machine learning model to provide real-time feedback for automatically controlling the advancement system 234. For example, the machine learning model may analyze the captured image data to determine how the inspection scope 30 should be advanced through the medical device M. In some embodiments, the machine learning model may output findings which are validated/approved by a user prior to reporting and/or storing in a database.

In some embodiments, a user manually advances the inspection scope 30 through a medical device M. Examples of the inspection scope 30 are disclosed herein. For example, the inspection scope 30 can be a borescope, such as a fiber scope. In some embodiments the inspection scope 30 includes one or more fiber optic elements (which can include one or more optical fibers, such as a fiber bundle) that carry light from a light source to the tip of the inspection scope 30. In other embodiments a light source (such as a light emitting diode (LED)) is positioned at or proximate the tip. Further, in some embodiments the fiber optic elements transmit light from the tip back to a camera 240 (or other optical sensors) located remote from the tip.

The camera 240 operates to capture images of the medical device M. The images can be individual images or video. The video can be composed of a plurality of images. The image and video data are included with the inspection data 232 which is transferred (either via a wired connection or wirelessly) to the computing device 104. The inspection data can also include time stamps identifying a date and/or time at which the images were taken. In some embodiments, the inspection data 232 also includes operational data. Examples of inspection data are disclosed herein.

The medical device M can be one of various different types of medical devices and may include an elongated flexible body with one or more internal orifices. Examples include endoscopes, fiber scopes, catheter-based medical/surgical instruments, and other reusable instruments.

Some embodiments include a position tracker 238. The position tracker 238 is configured to detect and monitor a position of the inspection scope 30 relative to the medical device M during the medical device inspection. Examples of the position tracker are described herein.

The computing device 104 operates the medical device inspection system 108. In one example, the medical device inspection system 108 includes an inspection analyzer 228 and an abnormality detector 230, examples of which are disclosed herein.

The computing device includes a display device 216 for presenting a user interface 224. In some embodiments, the outputs from the medical device inspection system 108 are presented on the display device 216. The user interface 224 can display images from the inspection alongside additional information, such as operation data or analysis data, or other displays based on the same. Examples of the user interface 224 are illustrated and described with reference to FIGS. 5-7. Other examples are described herein. In some embodiments, the user interface allows a user to reference a sequence of images in the order of the inspection (e.g., from the distal or proximal end).

In some embodiments the inspection system 20, including the medical device inspection system 108, and the inspection assembly 231, operates to perform inspections of one or more landmarks, such as particular points of interest of a medical device. The performance of the inspections may be automated, or in other embodiments, the inspection station 22 can provide instructions or otherwise guide an operator to inspect such landmarks. An example of a landmark is a hotspot. A hotspot is a point or area of a medical device that is prone to having abnormalities. In some embodiments hotspots are predetermined. A hotspot can be based on physical characteristics, or visually identifiable characteristics, such as a joint, transition, or intersection between two parts or materials, a recess or indentation, an opening, a surface texture, and the like. In some embodiments hotspots are identified from analysis of research or literature indicating spots where abnormalities are most likely. In some embodiments, the hotspots are identified by data that is updated from inspection software. For example, hotspots can be provided by other parties (or other systems) such as the FDA, manufacturers, third-party repair specialists, device cleaning specialists etc. In some embodiments, the hotspots are updated in real-time.

In some embodiments one or more landmarks can be identified. The landmarks may be predefined and stored in a database, such as in association with the type of medical device. For example, the landmarks can be linked to a specific medical device (serialized), linked to a make/model year, category of device, etc. landmarks can also be defined manually by an operator. For example, an operator can identify particular points on the medical device as landmarks or hotspots.

Various user interface configurations can be used to receive the identification of landmarks from the operator, such as by receiving inputs into a picture of the medical device M, into a diagram of the medical device M, or by providing position information (e.g., a length of 10 cm from a front end of the medical device, or a range from 5 cm to 15 cm from the front end of the medical device). In some embodiments, the landmarks are identified at a specific position identified by image recognition or by an end user. For example, water channels junctions, elevator mechanisms, distal tips may be recognized and identified from a captured image.

In yet another embodiment, landmarks can be determined automatically, such as by computer analysis of historical data to determine the most common areas where abnormalities have been previously identified for this type or model of medical device. Computer analysis can also happen on the fly, such as using artificial intelligence to automatically predict and identify landmarks for the medical device M such as current image data, knowledge of the structure of the medical device, and/or historical data for this or other similar medical devices.

In some embodiments, inspection system 20 is configured to present to the operator a tutorial of landmarks for a selected medical device M once the medical device M has been identified. The tutorial can include a training presentation that walks through the one or more landmarks, one or more diagrams of the medical device M with the landmarks identified, example inspection scope imagery showing the operator what it will look like during the inspection, or a variety of other possible training presentations or visual representations.

In some embodiments the inspection system 20 is configured to store and present or otherwise provide or make available historical records regarding, for example, the particular medical device M, the make/model, the category of device, and/or the age of the device. For example, historical photographs of the medical device M or medical device inspections can be shown to the operator or incorporated into a report. This can help the operator know about any known or previous abnormalities that were identified, and can provide reference imagery that the operator can use to compare the previous condition with the current condition. The historical records can include whether the device is new, the age of the device, the number of times the medical device has been used, recent or past damage, recent or past repairs, or other information.

In some embodiments, the information can also include patient data, such as information about what patient the medical device M was previously used with (e.g., the patient's name or a patient identification number), what procedure was performed, medical findings or diagnosis (such as to document that the medical device may have been exposed to certain biohazards, chemicals, radiation, or the like), or other patient-related data (with or without patient identifying information). The information can also include healthcare provider information, such as information about the medical professional(s) that last used the medical device M. The information can also include past (historical) patient or healthcare provider information.

The medical device inspection system 108 operates in some embodiments to coordinate the medical device inspection. For example, the medical device inspection system 108 can automatically control the inspection assembly 231 (including the advancement system 234 and the inspection scope 30) to perform the medical device inspection, such as using control signals 236. The medical device inspection system 108 can use retrieved information to identify landmarks within the medical device for inspection, and control the advancement system 234 so that the camera 240 obtains images of those areas. Other options are possible as well, as discussed herein, such as a complete inspection of the medical device M. The resulting inspection data 232 including the imagery can then be stored by the medical device inspection system 108, such as in the asset database 118.

In another example, the medical device inspection system 108 assists an operator in performing the medical device inspection. In this example, a user interface can be presented to guide the operator through the inspection. Certain operations may still be automatically controlled by the medical device inspection system 108, even when an operator is involved. Various information, guidance/instructions, reference imagery, etc. can be presented during the inspection to assist the operator, as discussed in further detail herein.

Analysis of the inspection data is then performed in some embodiments utilizing the inspection analyzer 228 and an abnormality detector 230. The inspection analyzer 228 processes the inspection data and can generate analysis results. In some embodiments the inspection analyzer 228 operates to identify landmarks in the medical device. In some embodiments the inspection analyzer 228 utilizes the position data generated by the position tracker 238. In some embodiments, the inspection analyzer utilizes one or more machine learning models to perform object recognition and identify landmarks of the medical device, for example.

To assist with the analysis, the inspection analyzer 228 utilizes the abnormality detector 230 in some embodiments. The abnormality detector 230 operates to evaluate the medical device to evaluate whether or not abnormalities may be present. In some embodiments the abnormality detector 230 may utilize human input, such as by displaying the corresponding image for a particular landmark, along with a reference image, and prompting the user to provide input on whether or not an abnormality is present at the landmark. In another example, the abnormality detector 230 utilizes a machine learning model to automatically analyze one or more images of the medical device, to determine or predict whether or not an abnormality may be present.

In some embodiments the abnormality detector 230 is or includes a neural network, such as a convolutional neural network (CNN). The neural network operates, in some embodiments, to process the image data from the medical device inspection, and extract features from the images, for example.

In some embodiments the abnormality detector 230 includes an input layer, that accepts the medical device images from the medical device inspection. In some embodiments the images are preprocessed. Preprocessing includes, for example, one or more of: resizing, normalization, augmentation, greyscale conversion, or noise reduction. Such preprocessing can improve the quality of the subsequent machine learning processing by providing consistent inputs into the model.

In some embodiments the abnormality detector 230 includes a plurality of convolutional layers. The layers are configured to detect patterns, textures, and features in the images, for example. Multiple layers can be combined with pooling layers to improve the ability of the abnormality detector 230 to understand different aspects of the images.

In some embodiments the abnormality detector 230 includes one or more fully connected (FC) layers. An FC layer is an example of a dense layer. One or more dense layers can be used to help the abnormality detector 230 make classification determinations. For example, the one or more FC layers can be used to combine extracted features and perform the final classification.

In some embodiments the abnormality detector 230 includes an output layer, which provides an output of the machine learning model. For example, the output layer can output a determination of whether or not the medical device has an abnormality. An example of the output is “normal” or “abnormal”. As another example, the output can include a probability (i.e., that the medical device is normal or abnormal), such as in the form of a percentage or a number from 0 to 1, for example. In some embodiments the output is binary (i.e., a binary classification by a binary classifier), while in other embodiments the output can have multiple outputs (i.e., a multi-class classification by a multi-class classifier).

In some embodiments, the abnormality detector 230 is trained using a labeled training set. In one example, the training involves a training algorithm, such as a gradient descent algorithm) to adjust weights of the neural network to minimize differences between its predictions and the actual labels.

Further, in some embodiments the abnormality detector 230 takes advantage of one or more reference images, allowing it to compare the medical device inspection data images to the reference images. In some embodiments, the abnormality detector 230 utilizes image differencing, in which a reference image (of a normal device without an abnormality) is subtracted from an inspection image to highlight differences. In some embodiments the abnormality detector 230 utilizes thresholding to convert the difference image to binary (black and white) to emphasize significant differences. In some embodiments the binary image can then be used as an additional input into the neural network to help the abnormality detector better focus on the differences.

In some embodiments the abnormality detector 230 can include continuous learning, such as a feedback loop and re-training. The feedback loop allows additional images (such as those containing abnormalities, or both normal and abnormal images) that are collected to be added to the training set. The model can then be re-trained on the updated data set to improve its accuracy.

In some embodiments, the output of the abnormality detector 230 is presented to an operator for review. The operator can make a final determination of whether or not an abnormality is present. In some embodiments the operator provides a user input to update the abnormality determination. In some embodiments the user input overrides (or confirms) an automatic abnormality determination by the abnormality detector 230. Further, in some embodiments the user input can be provided to manually identify an abnormality in the medical device that was not detected by the abnormality detector 230, which is then recorded.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

What is claimed is:

1. An asset tracking system comprising:
at least one processing device; and
at least one memory device storing asset tracking software and a medical device inspection system, wherein the medical device inspection system, when executed by the processing device, supplements the asset tracking software to provide additional functionality for inspection of a medical device using a medical device inspection system.

2. The asset tracking system of claim 1, wherein the medical device inspection system is a plugin to the asset tracking software.

3. The asset tracking system of claim 1, wherein the medical device inspection system is in data communication with an inspection system including at least an inspection scope, wherein the medical device inspection system receives and stores at least one image of an interior of the medical device captured by the inspection scope.

4. The asset tracking system of claim 1, wherein the medical device inspection system further comprises an inspection analyzer for analyzing inspection data from an inspection scope.

5. The asset tracking system of claim 1, wherein the memory further stores medical device inspection software, wherein the medical device inspection software includes the medical device inspection system and the asset tracking software.

6. The asset tracking system of claim 1, wherein the asset tracking software and the medical device inspection system are separate and wherein the asset tracking software and the medical device inspection system are configured to communicate with each other.

7. An asset tracking system comprising:
at least one processing device; and
at least one memory device, the memory device storing data instructions that, when executed by the processing device, cause the asset tracking system to:
identify a medical device;
retrieve a workflow for the medical device; and
track progress of the medical device through the workflow, wherein the workflow comprises at least one inspection step involving inspection of the medical device using an inspection scope.

8. The asset tracking system of claim 7, wherein the asset tracking system is further caused to:
retrieve at least one reference image associated with the medical device; and
generate a user interface that displays the reference image and an inspection image from the inspection scope.

9. A computer readable storage device storing data instructions for a medical device inspection system, which when the data instructions are executed by a processing device, cause the computing device to:
retrieve at least one reference image associated with the medical device; and
generate a user interface including the reference image and an inspection image from the medical device inspection scope within an asset tracking software application.

10. The computer readable storage device of claim 9, wherein the user interface includes one or more of:
(i) historical data about the medical device;
(ii) a location of the medical device;
(iii) an abnormality detected during inspection of the medical device;
(iv) a severity of the abnormality detected during inspection of the medical device;
(v) a status of the medical device;
(vi) a device identifier;
(vii) operator notes from the inspection of the medical device; or
(viii) any combination of (i)-(viii).
